(19) United States
(12) Patent Application Publication
(10) Pub. No.: US 2009/0299419 A1
(43) Pub. Date: Dec. 3, 2009

(54) Blood flow stimulation bandage

(75) Inventor: Scott Andrew West, Collingwood (CA)

Correspondence Address: Scott Andrew West, 241 Minnesota Street, Collingwood, ON, L9Y 3S4, CA

(73) Assignee: Scott Andrew West, Collingwood (CA)

(21) Appl. No.: 12/322,946

(22) Filed: Feb. 10, 2009

Related U.S. Application Data
(60) Provisional application No. 61/071,929, filed on May 27, 2008.

(51) Int. Cl. A61N 1/00; A61H 1/00; A61F 13/00

(52) U.S. Cl. 607/3; 601/2

(57) ABSTRACT

Flexible, form fitting, uniquely shaped, body part bandages incorporate flexible carbon fibre blood flow stimulation planar emitters to provide therapeutic blood flow stimulation. Emitters are electrically powered and radiate pulsed electro magnetic energy at an ideal wavelength—typically in the 0.8 micron to 15 micron range—and frequency—typically in the 0.5 Mhz to 5 MHz range or 20 Hz to 200 Hz range—to stimulate circulation in sub dermal tissue. The carbon fibre emitters provide the means to maximize and control surface coverage and through their planar design also provide very uniform energy emissions over the entire applied surface area.

Blood flow stimulation therapy body bandages are uniquely fashioned and shaped to form fit each body part and constructed such that they can then be incorporated in flexible and elastic materials such as neoprene and may include VELCRO hook and loop fasteners, buttons, straps, laces and the like.

### BACKGROUND OF THE INVENTION

This invention relates to radiant energy body part therapy devices, and more particularly to muscle and/or body part bandages incorporating electrically powered, flexible, carbon fibre, radiant energy blood flow stimulating planar emitters.

There is a long history for the treatment options for pain, to relax muscles and to increase blood circulation. Generally the intention is to promote vasodilatation of the blood vessels where the muscular inner walls of the blood vessels relax and the inner diameter widens. By promoting blood flow in an injured area, oxygen is delivered to the injured cells and cellular waste is carried away. Application of physical stimulation results in the production of hyperemia, induction of reflex vasodilatation, and acceleration of metabolic processes. Blood flow stimulating devices have been used for a variety of indications. General indications for therapeutic blood flow stimulating devices include pain, muscle spasm, contracture, tension myalgia, hematoma resolution, bursitis, tenosynovitis, fibrositis, fibromyalgia, superficial thrombophlebitis, and collagen vascular diseases.

Manual physical massage of tissue is the oldest form of stimulating blood flow. Fomentation devices attempt to stimulate blood flow using conductive heat as an alternative to physical massage. Acoustic, photo electric and direct electrical diathermy devices attempt to improve on penetration of stimulation past the dermal layer over Fomentation though the use of electrical charges or vibrational sound (ultrasound) waves or a light source. Infrared Radiant Energy is perhaps the most promising of all modalities.

The human body naturally generates and absorbs a spectrum of radiant energy. Radiant energy heats the body by conversion. Radiant energy penetrates the body and is absorbed by the cells. The cells vibrate and convert the radiant energy into heat energy.

To date, radiant energy blood flow stimulating was done with lamps, chemical packs, heating elements with IR filters using point source or linear elements. In 1979 Joseph M. bender proposed a method of applying therapeutic radiant energy in U.S. Pat. No. 4,303,074 that was issued Dec. 1, 1981. Bender's proposal was for a uniform, planar radiating pad that would greatly advance the art. Bender's invention, though, omitted a real limitation of the design in that it could not be practically applied to a non-uniform (rectangular) element or bandage design.

Bender's illustrations included with his patent illustrate the planar element's non-uniform wrapping of a body part. Complete wrapping of a body part in this fashion results in folding and overlapping of the element. This in turn causes several problems. Overlapping can create shorting of the electrical circuit resulting in a non-uniform resistance and resultant non-uniform heating pattern (hot spots). Internal stresses and non uniform compression along fold lines also produce changes in the conductivity properties of the proposed material again introducing defects in the real operation of the proposed design. Those familiar with the art will also recognize that an attempt to make a non-rectangular 1 piece heating element as proposed will again result in a non-uniform resistance pattern which defeats the desired uniform radiant properties of the proposed design.

Industry advancements have provided commercially available carbon fibre cloth that is now suitable of applications proposed in Bender's design. Even with the use of Carbon Fibre cloth, many of the limitations of Bender's invention remain. The proposed invention of this application greatly advances the art eliminating many for the design constraints of the Bender invention. Additionally this invention describes emitters that can be shaped and incorporated into form fitting bandages manufactured using flexible and/or elastic materials such as neoprene and fasteners such as VELCRO, buttons, hooks, laces or straps. This type of form fitting electrically powered radiant energy emitting body part wrap is previously unseen in the art.

Several other approaches to energy therapy treatments are know in the prior art including:

| US Patent Document No. | Inventor |
|---|---|
| US2008/0077203 A1 | Malak |
| US 2008/0156786 A1 | Choi |
| US 2008/0200971 A1 | Dodo |
| 4,303,074 | Bender |
| 4,960,979 | Nishimura |
| 4,981,135 | Hardy |
| 6,108,581 | Jung |
| US 6,458,109 B1 | Henley et al. |
| US 6,921,374 B2 | Augustine |

| Foreign Patent Document No. | Country & Date |
|---|---|
| DE 101 35 339 A1 | Germany, February 2003 |
| JP 7-085954 | Japan, March 1995 |
| JP 7-282961 | Japan, October 1995 |
| JP 8-38539 | Japan, February 1996 |
| JP 2001-137367 | Japan, May 2001 |

### BRIEF SUMMARY OF THE INVENTION

The invention describe herein advances the art. The carbon fibre emitter described herein provides a much broader range of shape possibilities. This new material provides the means to mold to complex body shapes. The electromagnetic properties of the carbon material also provide a means to standardize the energy output over the surface area of the emitter. This provides uniform treatment over the afflicted area and greatly reduces missed zones, low intensity zones, and high intensity zones (hot spots). The present invention is not intended to treat open wounds and therefore is not fashioned to retain moisture in the treatment area. The preferred embodiment includes a layer of nylon mess between the emitter and the skin. The mesh provides a means for surface heat and moisture to escape thereby helping to maintain the skin at a comfortable temperature.

The invention described herein uses a signal stage process to generate electromagnetic radiation in a preferred frequency range. Typical emitters use a broad spectrum heat source and use a layer of filter material to reflect or filter the unwanted frequencies. The carbon fibre energy emitters utilized in the present invention are highly efficient at converting electrical power in to electro magnetic radiation in the preferred range of frequency.

The energy therapy bandage utilizes a unique flexible blood flow stimulating emitter that radiates energy at an ideal frequency which penetrates deep into human tissue. The emitter is made with carbon fibre fabric laid out in a pattern and shaped and curved to mold to a body part. The patterned shape must always provide a uniform rectangular path for the current to flow in order to not introduce unbalance emissions (Hot Spots). This is achieved by forming the pattern using rectangular strips as broad in width as possible and laid out as close as possible without touching adjacent paths and connecting the individual strips using conductive materials such as adhesives or metal mesh. The resultant pattern can also be designed to optimize a desired Voltage to Length ratio to obtain optimal operational performance of the device. The resultant emitter material radiates energy evenly across its entire surface area. The emitter is housed in a form fitting compression wrap. This bandage works to focus the radiant energy device directly on the intended body part. The blood flow stimulating emitter radiates a majority of its energy with a wavelength between 3 and 15 microns. The bandage is fashioned with flexible and elastic material, combined with adjustable fasteners to apply the energy emitter. The device is powered electrically and uses hardware and software to control the output.

It is an object of the present invention to provide a radiant energy therapeutic wrap for body parts. It is another object of the present invention to provide radiant energy at an idea wavelength which is naturally absorbed by the body. It is another object of the present invention to provide a flexible radiant emitter fashioned such that the emitter wraps the body part and transmits energy efficiently and evenly. It is yet another object of the present invention to incorporate radiant energy treatment in an integral and consolidated device that is convenient to apply and use.

### BRIEF DESCRIPTION OF THE SEVERAL VIEWS OF THE DRAWING

FIG. 1A depicts a carbon emitter shaped and contoured to conform to a joint.

FIG. 1B illustrates a compression wrap shaped constructed to apply the emitter.

FIG. 1C shows a power supply and programmable power controller.

FIG. 1D shows a battery power supply connected to a programmable power controller.

FIG. 2A illustrates a carbon emitter shaped for the ankle joint.

FIG. 2B depicts a carbon emitter conforming to the ankle joint.

FIG. 2C depicts a carbon emitter shaped to conform to the knee joint.

FIG. 2D illustrates a carbon emitter conforming to the knee joint.

FIG. 3A depicts a carbon emitter shaped for the elbow joint.

FIG. 3B illustrates a carbon emitter curved to contour to the elbow joint.

FIG. 3C depicts a carbon emitter shaped for the shoulder joint.

FIG. 3D illustrates a carbon emitter conforming to the shoulder joint.

FIG. 4A depicts a carbon emitter shaped for the ankle joint.

FIG. 4B depicts a carbon emitter shaped for the knee joint.

FIG. 4C depicts a carbon emitter shaped for the lower back.

FIG. 4D depicts a carbon emitter shaped for the full length back.

FIG. 4E depicts a carbon emitter shaped for the upper back and neck.

FIG. 4F depicts a carbon emitter shaped for the shoulder joint.

FIG. 4H depicts a carbon emitter shaped for the wrist joint.

FIG. 5A is a diagram of a carbon emitter circuit and a method for fine adjustment.

FIG. 5B is a drawing detailing the radiant energy emission pattern.

FIG. 5C illustrates a typical element layout and the associated emission pattern.

FIG. 6A illustrates the details of the compression wrap designed to hold the emitter.

FIG. 6B show a compression wrap, housing an emitter and applied to a joint.

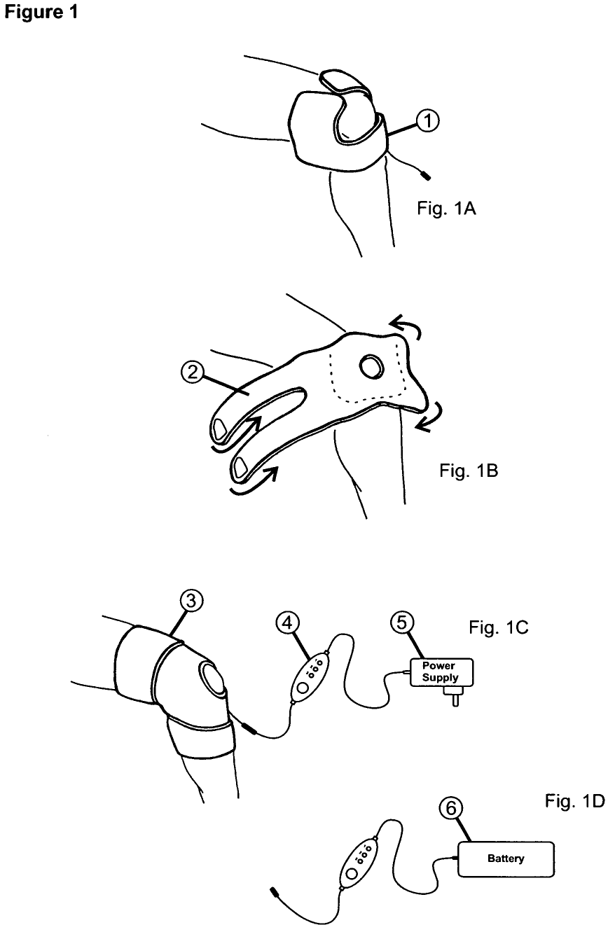

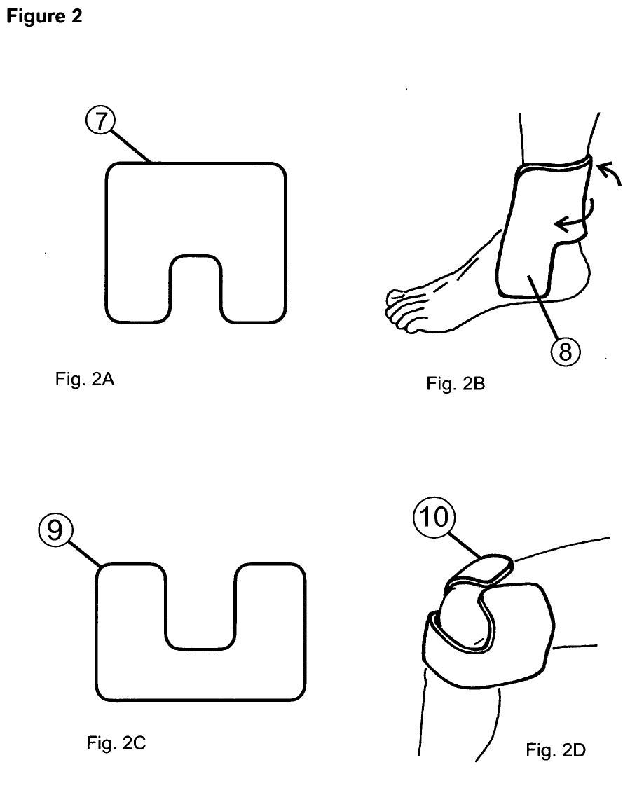

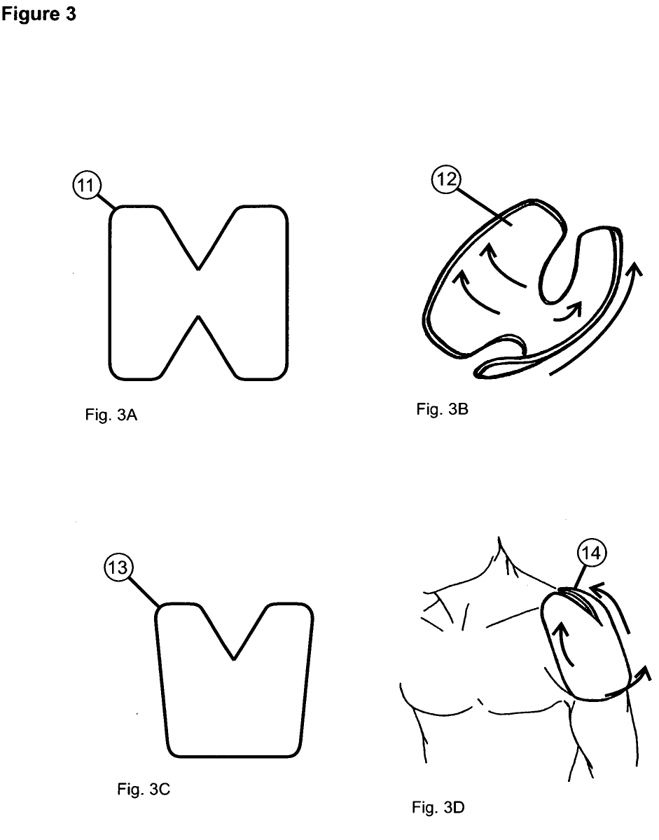

### DRAWING REFERENCE POINTS

1. Carbon emitter fashioned to contour to a joint without over lap.
2. Compression wrap fashioned to apply a carbon emitter to a joint
3. Compression wrap applied to a joint.
4. Programmable energy output controller for carbon emitter.
5. Power adapter to supply power from a home receptacle.
6. Battery power supply for the carbon emitter.
7. Carbon emitter shaped for the ankle joint.
8. Carbon emitter applied to the ankle joint.
9. Carbon emitter shaped for the knee joint.
10. Carbon emitter applied to the knee joint.
11. Carbon emitter shaped for the elbow joint.
12. Carbon emitter contoured for the elbow joint.
13. Carbon emitter shaped for the shoulder joint.
14. Carbon emitter applied to the shoulder joint.
15. Flexible conductor connection carbon emitter bands.
16. Minimized gap between carbon emitter bands.
17. Adhesive conductor to adjust the length of the carbon emitter circuit.
18. Carbon fibre emitter bands with specific output characteristics.
19. Energy emissions overlapping to provide uniform penetration.
20. Output energy pattern of carbon fibre emitter bands.
21. Gap in emission coverage with typical emitter configurations.
22. Energy emission pattern for typical emitter (existing technology).
23. Typical emitter cord element.
24. Shaped wrap fashioned to contour to a joint.
25. Elastic material for flexible conformity to the joint and compression.
26. Inside shaped pocket to house the energy emitter.
27. Versatile fastening device to secure the wrap in place.
28. Compression wrap shown applied to a joint.

### DETAILED DESCRIPTION OF THE INVENTION

There is a segment of the sun's energy spectrum which humans naturally absorb. This segment of radiant energy penetrates deep into the body. On a cold sunny day you can feel the warmth of radiant energy penetrating your body. It's cold outside but you feel warm inside. Radiant energy is a form of electromagnetic energy. Unlike conduction and convection, electromagnetic waves need no material medium for transmission. Radiant energy waves pass through some materials while being absorbed by others.

Radiant energy has the ability to penetrate and radiate in the body. The human body naturally absorbs radiant energy. Radiant energy penetrates the skin and radiates heat in the deeper tissues. This thermal affect within the deep layers of tissues can cause blood vessels in capillaries to dilate, promoting better blood circulation. The human body naturally generates and absorbs radiant energy at wavelengths between 2-50 microns. The body emits radiant energy concentrated between 8-10 microns. A majority of the energy radiated by the flexible blood flow stimulating emitter utilized in the described invention has a wavelength between 3-15 microns.

Oxygen (bound to haemoglobin in red blood cells) is the most critical nutrient carried by the blood. Damaged tissues need oxygen rich blood to heal. Injury and trauma cause blood vessels to constrict. Radiant energy promotes vasodilatation of the blood vessels. The muscular inner walls of the blood vessels relax and the inner diameter widens. By promoting blood flow in an injured or afflicted area, oxygen is delivered to the cells and cellular waste is carried away. Radiant energy heat therapy can effectively reduce healing time and aid in pain reduction.

The present invention provides a form fitting therapeutic compression wrap 2 incorporating a flexible carbon fibre radiant energy blood flow stimulating emitter 1 so that radiant energy heat therapy can be applied to body parts and adjacent tissues.

In the preferred embodiment, the radiant energy heater would consist of flexible carbon fibre fabric grid 7, be powered electrically 5 and would emit a majority of radiant energy at a wavelength between 3 and 15 microns.

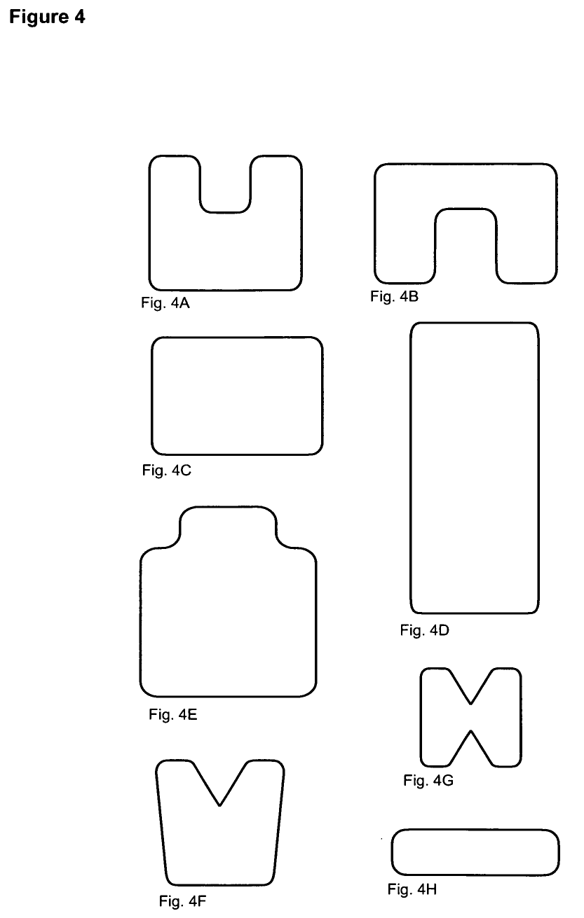

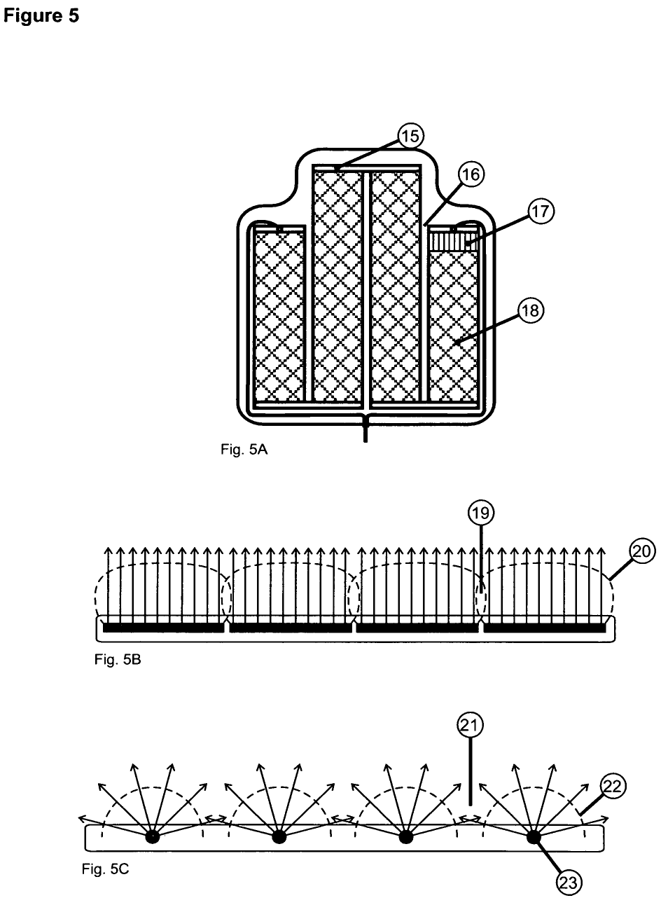

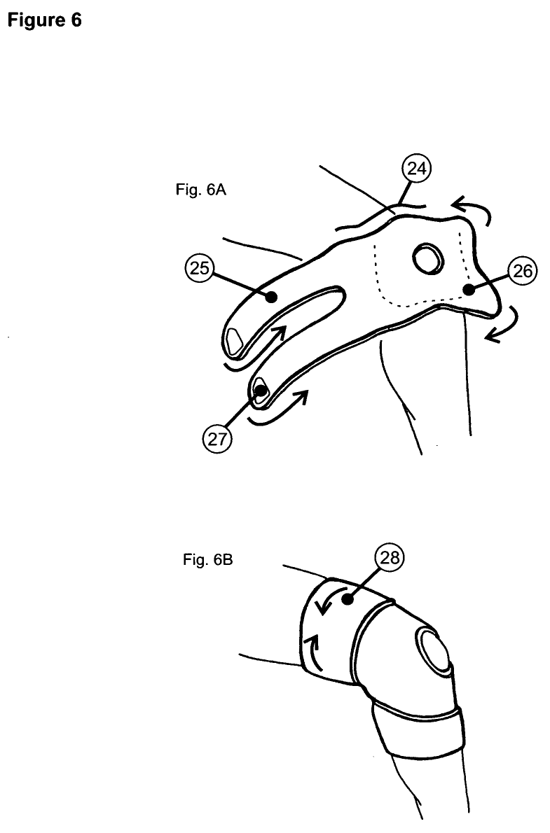

In order to better accommodate the body part, a unique emitter shape may fashioned—See FIG. 4. The shape incorporates a circuit constructed with the carbon fabric 18, flexible conductor cord 15 and conductive adhesive 17. Such an aperture may accommodate a body part such as a foot, ankle, knee, leg, back, shoulder, neck, elbow, wrist or the like. In the preferred embodiment a careful balanced is maintained in the ratio of volts to the total length of the carbon fabric circuit. The preferred ratio is between 5 volts per meter and 24 volts per meter with a most preferred design setting of 15 volts per meter. Circuits, as defined by one or more rectangular strips of carbon fibre fabric connected to each other with conductive couplers, are designed to accommodate a close fit the body part. The supply voltage and the length of the circuit are adjusted to provide optimal intensity of radiate energy. The specific length of the circuit may be finely adjusted using conductive adhesive to effectively shorten the semi-conductive material at any point and in any form thus maintaining a uniform conductivity path which in turn provides uniform energizing of the carbon fibre material. The runs of the carbon circuit can be very close together providing uniform energy output across the entire surface of the emitter—FIG. 5B.

Additionally, in order best apply the heat emitter to the body part a form fitting compression wrap 2 may be fashioned 24 utilizing a flexible material such as neoprene 25 and may be fastened using attachment devices such as VELCRO hook and fasteners 27, or the like. Buttons, snap, straps and other fasteners may also be used to good advantage.

The compression bandage includes a compartment 26 to house the emitter. The emitter can be removed from the bandage. The position of the emitter can be adjusted inside the compartment.

The emitter is powered by an external power supply 5. The power signal to said emitter is varied in one or a plurality of amplitude, frequency, modulation or other electrical characteristics to alter one or a plurality of the operation intensity, frequency, or pulse rate of the emitted blood flow stimulating radiant energy. The emitter operates with variable outputs selectable through dials or switches 4 on the device or the power supply or through software or otherwise. The device can also be powered from a mobile power source such as a battery 6 or regenerative power supply.

1. An electrically powered radiant energy emitting therapeutic bandage for body parts, comprising: one or more uniquely shaped, carbon fiber, radiant energy emitting elements defining an emitter; said emitter accommodating and conforming to a human body part shape such that the emitter does not overlap itself nor fold over its own surface while maintaining a uniform path of conductivity for even energy transfer and flow, providing an even and uniform covering of the body surface and uniform emission of energy over the entire surface, at all times radiating energy perpendicular to the surface of the underlying body part; attachable detachable flexible elastic compression bandage housing such that said blood flow stimulation emitter is fashioned to adapt the blood flow stimulation emitter to a limb or body part whereby said body part may be subject to radiant energy therapy.

2. The device according to claim 1 wherein said emitter is shaped such that when wrapped around said body part the emitter does not fold or overlap upon itself.

3. The device according to claim 1 wherein said emitter is laid out in a pattern to provide uniform radiant energy emission over the entire surface.

4. The device according to claim 1 wherein said emitter is laid out in a pattern to provide a non-uniform but intended energy emission pattern over the entire surface.

5. The device according to claim 1 wherein said emitter is powered by an external power supply.

6. The device according to claim 1 wherein the power signal to said emitter is varied in one or a plurality of amplitude, frequency, modulation or other electrical characteristics to alter one or a plurality of the operation intensity, frequency, or pulse rate of the emitted blood flow stimulating radiant energy.

7. The device according to claim 1 wherein the device also includes mechanical, vibration stimulation, ultrasonic stimulation, thermal stimulation or other combinational therapeutic modalities in addition to the blood flow stimulation of said device.

8. The device according to claim 1 for use on animals

9. The device according to claim 1 for use internally

10. The device according to claim 1 wherein the device is powered from a fixed power source such as a wall receptacle.

11. The device according to claim 1 wherein the device is powered from a mobile power source such as a battery or regenerative power supply.

12. The device according to claim 1 wherein the emitter operates at a fixed output.

13. The device according to claim 1 wherein the emitter operates with variable outputs selectable through dials or switches on the device or the power supply or through software or otherwise.

14. The device according to claim 1 wherein said emitter emits the majority of its radiant energy with a wavelength between 0.8-15 microns.

15. The device according to claim 1 wherein said emitter modulates the majority of it's radiant energy with a frequency between 0.5 MHz-5 MHz.

16. The device according to claim 1 wherein said emitter modulates the majority of it's radiant energy with a frequency between 20 Hz-200 Hz.

17. The device according to claim 1 wherein said radiant energy emitter is housed in a form fitting compression bandage which may be fashioned utilizing a flexible material such as neoprene and may be fastened using attachment emitters such as VELCRO hook and fasteners, buttons, snaps, straps and other fasteners may also be used to good advantage

18. The device according to claim 1 wherein said compression bandage includes a pocket to house the radiant energy emitter, whereby said emitter can be removed from the bandage.
